(19) United States
(12) Patent Application Publication
(10) Pub. No.: US 2026/0364073 A1
(43) Pub. Date: Nov. 19, 2026

(54) MAVACAMTEN AND DERIVATIVES THEREOF FOR USE IN TREATING ATRIAL DYSFUNCTION

(71) Applicant: MyoKardia, Inc., Brisbane, CA (US)

(72) Inventors: Carlos Del Rio, Brisbane, CA (US); Jay Edelberg, Brisbane, CA (US); Robert McDowell, Brisbane, CA (US)

(21) Appl. No.: 19/125,461

(22) PCT Filed: Nov. 1, 2023
(86) PCT No.: PCT/US2023/036587

§ 371 Date: Apr. 29, 2025

Related U.S. Application Data
(60) Provisional application No. 63/381,966, filed on Nov. 2, 2022.

(51) Int. Cl. A61K 31/513; A61P 9/06

(52) U.S. Cl. CPC A61K 31/513; A61P 9/06

(57) ABSTRACT

Provided herein are methods, uses, and compositions for treating atrial fibrillation in a patient, such as a patient exhibiting hypertrophic cardiomyopathy.

### CROSS-REFERENCE TO RELATED APPLICATION

This application claims priority to U.S. provisional application No. 63/381,966, filed Nov. 2, 2022, the entire contents of which are incorporated herein by reference.

### BACKGROUND OF THE INVENTION

Atrial fibrillation (AF) is the most common type of cardiac arrhythmia, affecting more than 37 million people worldwide. As the global population ages, the prevalence of AF is expected to increase. Patients with AF are at increased risk for stroke, cognitive decline, and cardiovascular events and mortality. AF is associated with underlying disorders such as hypertension, coronary heart disease, rheumatic heart disease, heart failure, obesity, diabetes mellitus, and chronic kidney disease. Symptoms include, but are not limited to, heart palpitations, tachycardia, shortness of breath, weakness, dizziness, fatigue, chest pain, and confusion.

AF is defined as a supraventricular tachyarrhythmia with uncoordinated atrial activation leading to ineffective atrial contraction, and can result from structural and/or electrical abnormalities of the atrium. Electrocardiographic characteristics include irregular R-R intervals (when AV conduction is present), no distinct repeating P waves, and irregular atrial activity. Episodes often increase in frequency and duration over time and become less responsive to medication. There are generally four types of AF (January et al., JACC (2014) 64(21):2246-80; Kirchhof et al., Eur Heart J. (2016) 37:2893-2962). Paroxysmal AF, also known as intermittent or self-terminating AF, terminates within seven days of onset, either spontaneously or with intervention. Persistent AF is continuous AF that is sustained for more than seven days; pharmacologic or electrical cardioversion may be required to restore sinus rhythm. Long-standing persistent AF is continuous AF that is sustained for more than 12 months, and may not respond to medication or cardioversion. Permanent (chronic) AF is persistent AF where the patient and the doctor jointly decide to stop further attempts to restore and/or maintain sinus rhythm.

AF impacts left atrial function and geometry, and vice-versa. AF is accompanied by a conversion from the fast a-myosin heavy chain (MHC) isoform to the slow β-MHC isoform (Narolska et al., Journal of Muscle Research and Cell Motility (2005) 26:39-48; Belus et al., Circulation Research (2010):107(1):144-52; Barth et al., Circulation Research (2005) 96(9):1022-9); Eiras et al., Journal of Molecular and Cellular Cardiology (2006) 41:467-77), reducing the speed of atrial contraction and relaxation. Over time, AF can result in decreased left atrial (LA) function (e.g., LA emptying fraction (LAEF)), as well as atrial remodeling (e.g., fibrosis and/or an increase in LA volumes that may become irreversible). Moreover, impaired LA function (e.g., LAEF) is associated with new-onset atrial fibrillation (Hirose et al., Eur Heart J. (2012) 13(3):243-50) as well as recurrence of AF after corrective procedures such as ablation. LA enlargement is strongly correlated with AF recurrence after electrical cardioversion. Impaired LA functional index (LAFI), calculated from LAEF, indexed maximal LA volume, and left ventricular outflow tract velocity time integral, is associated with adverse atrial remodeling, and increases the risk of developing incident AF and/or cardiovascular disease even when left atrial size is normal (Sardana et al., J Am Soc Echocardiogr. (2017) 30(9):904-12). LA parameters have been shown in observational studies to be powerful independent predictors of cardiovascular outcomes, including AF (Von Jeinsen et al., J Am Soc Echocardiograph. (2019) 33(1):72-81; Schaaf et al., Eur Heart J Cardiovasc Imaging (2017) 18:46-53).

AF is the most common arrhythmia in hypertrophic cardiomyopathy (HCM), and can cause significant impairment in the hearts of HCM patients. In some cases, AF can rapidly exacerbate HCM symptoms such as exertional dyspnea. Genetic (heritable) HCM comprises a group of highly penetrant, monogenic, autosomal dominant myocardial diseases. HCM can be caused by one or more of over 1,000 known point mutations in any one of the proteins contributing to the functional unit of myocardium, the sarcomere. About 1 in 500 individuals in the general population are found to have left ventricular hypertrophy unexplained by other known causes (e.g., hypertension or valvular disease), and many of these can be shown to have HCM, once other heritable (e.g., lysosomal storage diseases), metabolic, or infiltrative causes have been excluded. While many patients with HCM report minimal or no symptoms for extended periods of time, HCM is a progressive disease with a significant cumulative burden of morbidity, and has been associated with stroke, heart failure, and sudden cardiac death. In approximately 60% of patients with HCM, the left ventricular outflow tract (LVOT) becomes obstructed (obstructive HCM or oHCM), impeding the flow of blood and creating a pressure gradient between the LV cavity and the aorta. In other patients, the thickened heart muscle does not block the LVOT, and disease is driven by diastolic impairment due to the enlarged and stiffened heart muscle (non-obstructive HCM or nHCM). Patients with combined AF and HCM are at increased risk of stroke, heart failure, and death.

Current therapies for AF include rate and rhythm control strategies and corrective procedures such as surgery (e.g., ablation) and sinus rhythm-restoring cardioversion. However, impaired LA function and geometry contribute to AF recurrence after corrective treatment; no current therapies directly address both depressed atrial function and atrial enlargement. Accordingly, there remains a high medical need for new safe, well-tolerated, effective therapies for improving the atrial function of patients with AF, optionally when paired with other forms of cardiac dysfunction such as diastolic dysfunction (e.g., hypertrophic cardiomyopathy).

### SUMMARY OF THE INVENTION

The present disclosure provides a method of treating atrial dysfunction in a patient in need thereof, comprising administering to the patient a therapeutically effective amount of a compound of formula I below (I) (Compound I),

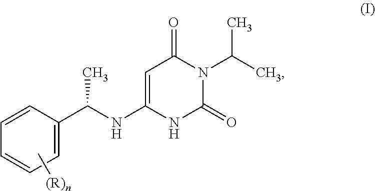

or a pharmaceutically acceptable salt thereof, wherein n is 0, 1, 2, 3, 4, or 5, and R is a halogen (e.g., fluorine (F)), optionally wherein the patient exhibits atrial fibrillation.

The present disclosure further provides a method of treating atrial cardiomyopathy in a patient in need thereof, comprising administering to the patient a therapeutically effective amount of a compound of formula (I),

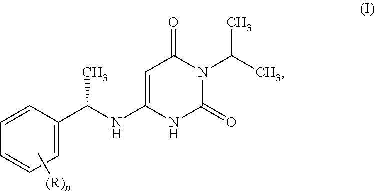

or a pharmaceutically acceptable salt thereof, wherein n is 0, 1, 2, 3, 4, or 5, and R is a halogen (e.g., F), optionally wherein the patient exhibits atrial fibrillation.

The present disclosure further provides a method of treating atrial tachyarrhythmia in a patient in need thereof, comprising administering to the patient a therapeutically effective amount of a compound of formula (I),

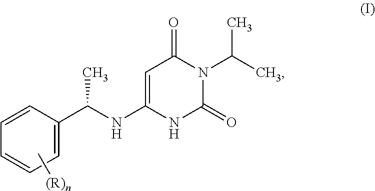

or a pharmaceutically acceptable salt thereof, wherein n is 0, 1, 2, 3, 4, or 5, and R is a halogen (e.g., F), optionally wherein the patient exhibits atrial fibrillation.

The present disclosure further provides a method of treating atrial fibrillation in a patient in need thereof, comprising administering to the patient a therapeutically effective amount of a compound of formula (I)

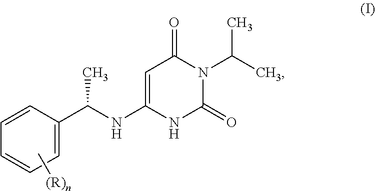

or a pharmaceutically acceptable salt thereof, wherein n is 0, 1, 2, 3, 4, or 5, and R is a halogen (e.g., F).

The present disclosure further provides a method of reducing atrial fibrillation recurrence in a patient in need thereof, comprising administering to the patient a therapeutically effective amount of a compound of formula (I),

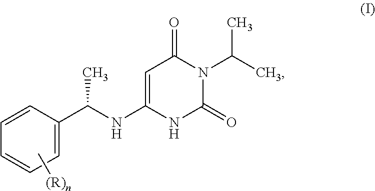

or a pharmaceutically acceptable salt thereof, wherein n is 0, 1, 2, 3, 4, or 5, and R is a halogen (e.g., F), optionally wherein atrial fibrillation recurrence is reduced by 10% or greater.

The present disclosure further provides a method of reducing atrial fibrillation burden in a patient in need thereof, comprising administering to the patient a therapeutically effective amount of a compound of formula (I)

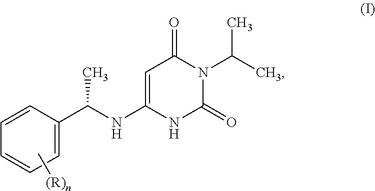

or a pharmaceutically acceptable salt thereof, wherein n is 0, 1, 2, 3, 4, or 5, and R is a halogen (e.g., F), optionally wherein atrial fibrillation burden is reduced by 10% or greater.

The present disclosure further provides a method of reducing the duration of an atrial fibrillation episode in a patient in need thereof, comprising administering to the patient a therapeutically effective amount of a compound of formula (I)

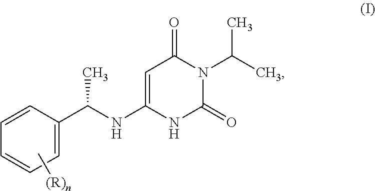

or a pharmaceutically acceptable salt thereof, wherein n is 0, 1, 2, 3, 4, or 5, and R is a halogen (e.g., F), optionally wherein the duration of the episode is reduced by 10% or greater.

The present disclosure further provides a method of reducing the number of atrial fibrillation episodes during a monitoring period in a patient in need thereof, comprising administering to the patient a therapeutically effective amount of a compound of formula (I)

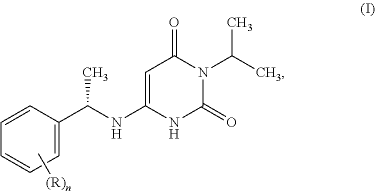

or a pharmaceutically acceptable salt thereof, wherein n is 0, 1, 2, 3, 4, or 5, and R is a halogen (e.g., F), optionally wherein the number of atrial fibrillation episodes is reduced by 10% or greater.

The present disclosure further provides a method of maintaining sinus rhythm in a patient in need thereof, comprising administering to the patient a therapeutically effective amount of a compound of formula (I)

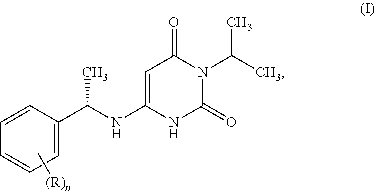

or a pharmaceutically acceptable salt thereof, wherein n is 0, 1, 2, 3, 4, or 5, and R is a halogen (e.g., F), optionally wherein the patient has sustained atrial tachyarrhythmia for 12 months or less prior to the administering step, further optionally wherein the atrial tachyarrhythmia is atrial fibrillation.

The present disclosure further provides a method of restoring sinus rhythm in a patient exhibiting atrial tachyarrhythmia, comprising administering to the patient a therapeutically effective amount of a compound of formula (I)

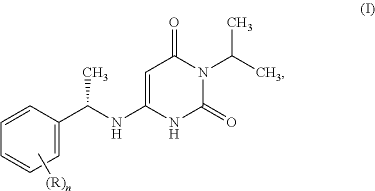

or a pharmaceutically acceptable salt thereof, wherein n is 0, 1, 2, 3, 4, or 5, and R is a halogen (e.g., F), optionally wherein the cardioversion is electrical cardioversion and further optionally wherein the atrial tachyarrhythmia is atrial fibrillation.

The present disclosure further provides a method of preventing or slowing the conversion of α-myosin heavy chain (MHC) to β-MHC in the left atrium of a patient, comprising administering to the patient a therapeutically effective amount of a compound of formula (I)

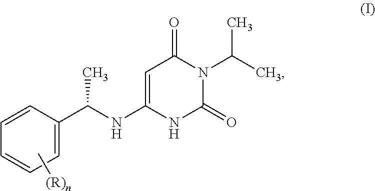

or a pharmaceutically acceptable salt thereof, wherein n is 0, 1, 2, 3, 4, or 5, and R is a halogen (e.g., F), optionally wherein the patient exhibits atrial fibrillation.

The present disclosure further provides a method of reversing the conversion of α-myosin heavy chain (MHC) to β-MHC in the left atrium of a patient, comprising administering to the patient a therapeutically effective amount of a compound of formula (I)

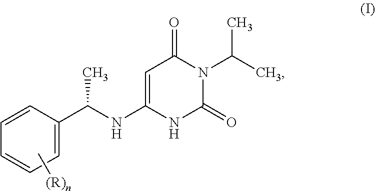

or a pharmaceutically acceptable salt thereof, wherein n is 0, 1, 2, 3, 4, or 5, and R is a halogen (e.g., F), optionally wherein the patient exhibits atrial fibrillation.

In the methods described herein, in some embodiments of formula I, n is 0. In other embodiments, n is 1. In some embodiments of formula I, R is fluorine. In some embodiments, R is a fluorine at position 3 (meta position).

In some embodiments of the methods described herein, the patient exhibits diastolic dysfunction, e.g., hypertrophic cardiomyopathy (HCM). The HCM can be obstructive hypertrophic cardiomyopathy (oHCM), and in certain embodiments the patient has at least a 30 mm Hg pressure gradient across the left ventricular outflow tract (LVOT) at rest, during or immediately after a Valsalva maneuver, or post-exercise. Alternatively, the HCM can be non-obstructive hypertrophic cardiomyopathy (nHCM).

In some embodiments of the methods described herein, the patient has heart failure and a diagnosis of any one of NYHA Class II-IV.

In some embodiments of the methods described herein, the patient exhibits atrial fibrillation, and the therapeutically effective amount of Compound I alleviates one or more symptoms of HCM, maintains sinus rhythm, reduces atrial fibrillation recurrence, and/or prevents incident atrial fibrillation in the patient.

The present disclosure also provides a method of preventing tachycardia-induced cardiomyopathy in a patient exhibiting atrial fibrillation, comprising administering to the patient a therapeutically effective amount of a compound of formula (I)

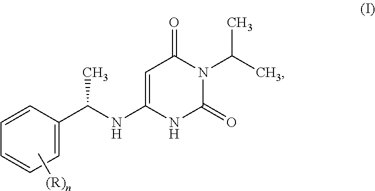

or a pharmaceutically acceptable salt thereof, wherein n is 0, 1, 2, 3, 4, or 5, and R is a halogen (e.g., F).

In some embodiments of the methods described herein, the patient has sustained tachyarrhythmia or atrial fibrillation for a duration of 12 months or less prior to the step of administering Compound I.

In some embodiments of the methods described herein, the patient has an atrial fibrillation burden of 2-70%.

In some embodiments of the methods described herein, the Compound I is administered to the patient orally.

In some embodiments of the methods described herein, the patient has undergone an electrical cardioversion before or after the step of administering Compound I, optionally where the electrical cardioversion is performed no more than 24 hours before or after the administering step.

In some embodiments of the methods described herein, an additional medication is administered for improving cardiovascular conditions in the patient.

In some embodiments of the methods described herein, the method results in any one or combination of the following:
a) reduced risk of urgent outpatient intervention for atrial dysfunction, diastolic dysfunction, or both;
b) improved quality of life as measured through 6-MWT or KCCQ;
c) improved exercise capacity;
d) improvement in a patient's NYHA classification;
e) delay in clinical worsening;
f) reduction in severity of cardiovascular-related symptoms;
g) increased left atrial ejection fraction (LAEF);
h) decreased left atrial volume (LAₘᵢₙVI);
i) improved left atrial function index (LAFI);
j) reduced cTnT leakage;
k) decreased BNP; and
l) decreased expression of pro-fibrotic genes.

In some embodiments of the methods described herein, the method results in reduced cardiovascular death, hospitalization, stroke, embolism, heart failure, or any combination thereof.

The present disclosure also provides a compound of formula (I)

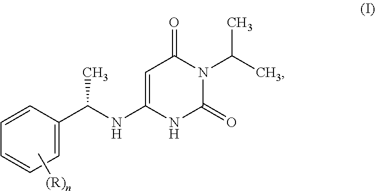

or a pharmaceutically acceptable salt thereof, wherein n is 0, 1, 2, 3, 4, or 5, and R is a halogen (e.g., F), or a pharmaceutical composition comprising the compound and a pharmaceutically acceptable excipient, for use in a method described herein and/or for use in treating a disease as described herein.

The present disclosure also provides the use of a compound of formula (I)

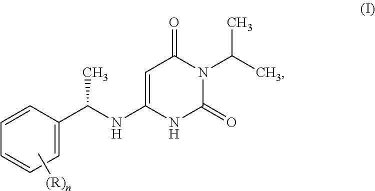

or a pharmaceutically acceptable salt thereof, wherein n is 0, 1, 2, 3, 4, or 5, and R is a halogen (e.g., F), in the manufacture of a medicament for treating a patient in a method described herein and/or for treating a disease as described herein.

Also provided in the present disclosure are pharmaceutical compositions comprising Compound I and a pharmaceutically acceptable excipient (e.g., for use in any of the treatment methods described herein); Compound I and the pharmaceutical compositions for use in any of the treatment methods described herein; and the use of Compound I for the manufacture of a medicament for use in any of the treatment methods described herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic diagram showing the design for an experiment on the chronic effects of Compound A on HCM and on left atrial and left ventricular geometry and function in a mini-pig model of inherited HCM (“HCM pigs”). Compound A dose is increased over the first 12 weeks of the study to account for weight gain. CMPD A: Compound A. CTRL: control. cMR: cardiac MRI. PV: LV pressure and volume studies (invasive hemodynamics). RNA: LV gene expression studies. HIST: histology. BIO: LV/LA fiber biomechanical studies.

FIG. 2 depicts RNA expression levels in LV gene expression studies in HCM pigs. CMPD A: Compound A. CTRL: control,

FIG. 3 is a set of graphs showing the effects of Compound A on left atrial (LA) geometry and function in HCM pigs. Compound A (CMPD A) decreased LA volumes (left panel), prevented the conversion of α-MHC to β-MHC in the LA (middle panel), and preserved isometric tension redevelopment dynamics (Kₜᵣ) (right panel), in comparison to untreated HCM pig controls (CTRL).

FIG. 4 is a graph showing the myosin ATPase inhibitory effect of Compound A and mavacamten in bovine cardiac myofibrils at pCa 6.0. The compounds demonstrate similar inhibition of myosin over the range of tested concentrations. CMPD: compound.

FIG. 5 is a graph showing the effects of Compound A and mavacamten in bovine cardiac myofibrils on disordered relaxed state (DRX) and super-relaxed state (SRX) ATPase rates.

### DETAILED DESCRIPTION OF THE INVENTION

The present disclosure provides methods, uses, and compositions relating to treating patients with atrial dysfunction (e.g., AF), including patients with comorbid atrial dysfunction and diastolic dysfunction (impairment of the diastolic function of the heart; e.g., due to hypertrophic cardiomyopathy (HCM), including both obstructive and non-obstructive HCM.

### Pharmaceutical Compositions

The pharmaceutical compositions used in the present therapies contain Compound I as an active pharmaceutical ingredient (API). Compound I has the following chemical structural formula (I):

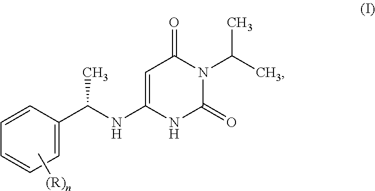

or a pharmaceutically acceptable salt thereof, wherein R is a halogen (i.e., fluorine, chlorine, bromine, or iodine) and n is 0, 1, 2, 3, 4, or 5. In some embodiments, n is 0, and the compound is mavacamten with the following formula:

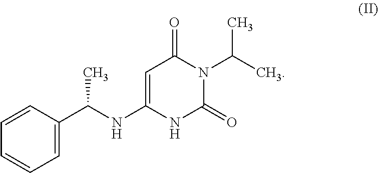 [SMILES: CC(C)n1c(=O)cc(N[C@@H](C)c2ccccc2)[nH]c1=O]

In some embodiments, R is fluorine and n is 1 or 2. In some embodiments, n is 1 and R is a fluorine at position 3 (i.e., meta position), and the compound is termed Compound A, with the following formula:

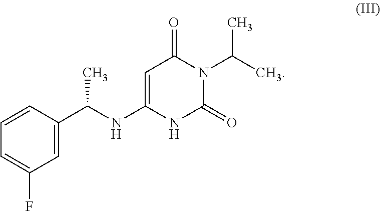 [SMILES: CC(C)n1c(=O)cc(N[C@@H](C)c2cccc(F)c2)[nH]c1=O]

Compounds of formula (I) (collectively “Compound I” herein) are selective allosteric inhibitors of cardiac myosin ATPase that can normalize the function of myosin in hypercontractile hearts. Because Compound I restores the contractile properties and energy demands of the heart, it may offer benefits such as prevention of left atrial remodeling.

In some embodiments, Compounds of Formula (I), (II) or (III) may be in the form of a pharmaceutically acceptable salt. In other embodiments, Compounds of Formula (I), (II) or (III) may be in the form of a free base.

The pharmaceutical compositions used herein may be provided in an oral dosage form (e.g., a liquid, a suspension, an emulsion, a capsule, or a tablet). In some embodiments, Compound I particles are compressed into tablets or capsules each containing 1, 2, 2.5, 5, 7.5, 10, 15, 20, or 25 mg of Compound I. In some embodiments, Compound I particles may be suspended in a suitable liquid such as water, a suspending vehicle, and/or flavored syrup for oral administration.

Besides the Compound I API, the pharmaceutical compositions of the present disclosure may also contain pharmaceutically acceptable excipients. For example, the tablets used herein may contain bulking agents, diluents, binders, glidants, lubricants, and disintegrants. In some embodiments, Compound I tablets contain one or more of microcrystalline cellulose, lactose monohydrate, hypromellose, croscarmellose sodium, and magnesium stearate. The tablets may be coated to make them easier to ingest.

### Patient Populations

The therapies of the present disclosure may be used to treat a patient exhibiting atrial dysfunction. For example, the patient may exhibit atrial fibrillation. Abnormal atrial contractility, volume, function, and/or atrial cardiomyopathy may contribute to the atrial dysfunction.

The atrial dysfunction being treated includes, without limitation, atrial cardiomyopathy and atrial arrhythmia (e.g., atrial tachyarrhythmia) such as AF or atrial flutter. The atrial dysfunction (e.g., atrial tachyarrhythmia) may be acute or chronic. In certain embodiments, the patient may have sustained the atrial dysfunction (e.g., atrial tachyarrhythmia such as AF) continuously for a duration of, e.g., no more than 10 years, 9 years, 8 years, 7 years, 6 years, 5 years, 4 years, 3 years, 2 years, 12 months, 9 months, 6 months, 3 months, 1 month, 2 weeks, or 1 week prior to a therapy of the present disclosure.

In some embodiments, the patient has AF, which may be clinically manifested or may be subclinical (asymptomatic). Where AF cases are caused by a heart valve disorder, they are termed valvular AF. AF without a diagnosed heart valve disorder is called non-valvular AF. For example, in some embodiments non-valvular AF is AF in the absence of rheumatic mitral stenosis, a mechanical or bioprosthetic heart valve, or mitral valve repair. In terms of timing and duration, the AF being treated may be, e.g., paroxysmal, persistent, or long-standing persistent. In some cases, the AF is persistent but not long-standing persistent AF; that is, it has been sustained for 12 months or less. In certain embodiments, the patient has an AF burden of 1-70%, 2-70%, 3-70%, 1-99%, 2-99%, etc. Unless otherwise indicated, AF burden refers to the amount of AF that an individual has. In some embodiments, AF burden may be defined as the percentage of time in which a patient is in AF during a monitoring period. In some embodiments, AF burden may be defined as the duration of a patient's longest AF episode, or the number of AF episodes during a monitoring period.

In some embodiments, the patient has a genetic predisposition to AF, such as an inherited cardiomyopathy or channelopathy.

In some embodiments, the patient has an implanted device with an atrial lead (e.g., pacemaker, ICD, CRT), or an implantable loop recorder (ILR).

In some embodiments, the patient has a Modified European Heart Rhythm Association (EHRA) symptom score of 1, 2a, 2b, 3, or 4, as defined in Table 1 below.

TABLE 1
Modified EHRA Symptom Scale

| Modified | | |
|---|---|---|
| EHRA | Patient | |
| Score | Symptoms | Description |
| 1 | None | AF does not cause any symptoms |
| 2a | Mild | Normal daily activity not affected by symptoms |
| | | related to AF |
| 2b | Moderate | Normal daily activity not affected by symptoms |
| | | related to AF, but patient troubled by symptoms |
| 3 | Severe | Normal daily activity affected by symptoms |
| | | related to AF |
| 4 | Disabling | Normal daily activity discontinued |

In some embodiments, the patient has been or is being treated with an anticoagulant, a rate control agent, a rhythm control agent, or any combination thereof, but continues to exhibit AF symptoms. Such symptoms may include, e.g., heart palpitations, tachycardia, fatigue, dizziness, weakness, chest discomfort, reduced exercise capacity, increased urination, shortness of breath, angina, presyncope, syncope, sleeping difficulties, confusion, and psychosocial distress, or any AF symptom described herein.

In some embodiments, the patient also exhibits diastolic dysfunction. Diastolic dysfunction may include, e.g., impaired left ventricle relaxation, filling, or diastolic distensibility, or left ventricle stiffness. In some embodiments, these traits may be measured using echocardiography. The diastolic dysfunction may be symptomatic or asymptomatic.

Diastolic dysfunction is present or an important feature of a series of diseases including, but not limited to, hypertrophic cardiomyopathy (HCM), left ventricular hypetrophy (LVH)—including malignant LVH, heart failure with preserved ejection fraction (HFpEF)—including both disorders of active relaxation and disorders of chamber stiffness (diabetic HFpEF), dilated cardiomyopathy (DCM), ischemic cardiomyopathy, cardiac transplant allograft vasculopathy, restrictive cardiomyopathy—including inflammatory subgroups (e.g., Loefilers and EMF), infiltrative subgroups (e.g., amyloid, sarcoid and XRT), storage subgroups (e.g., hemochromatosis, Fabry and glycogen storage disease), idiopathic/inherited subgroups such as Trop I (β-myosin HC), Trop T (α-cardiac actin) and desmin related (usually includes skeletal muscle), congenital heart disease subgroups (including pressure-overloaded RV, Tetrology of Fallot (diastolic dysfunction pre-op and early post-op, systolic dysfunction post-op) and pulmonic stenosis), and valvular heart disease (e.g., aortic stenosis—including elderly post AVR/TAVR and congenital forms).

In certain embodiments, the patient exhibiting atrial dysfunction also exhibits hypertrophic cardiomyopathy (HCM). Hypertrophic cardiomyopathy (HCM) is defined clinically as unexplained left ventricular (LV) hypertrophy in the absence of known causes such as pressure overload, systemic diseases, or infiltrative processes. The phenotypic hallmark of HCM is myocardial hypercontractility accompanied by reduced LV compliance, reflected clinically as reduced ventricular chamber size, often supranormal ejection fraction, increased wall thickness, and diastolic dysfunction. Some of the symptoms that HCM patients have include, but are not limited to, shortness of breath (especially during exercise), chest pain (especially during exercise), fainting (especially during or just after exercise), sensation of rapid, fluttering or pounding heartbeats, and heart murmur.

In certain embodiments, HCM is obstructive HCM. It is generally understood that obstructive HCM (oHCM) is defined as at least a 30 mm Hg (i.e., 30 mm Hg or higher) pressure gradient across the LVOT in an individual at rest, during or immediately after Valsalva maneuver, or post-exercise. In some embodiments, an individual with oHCM has an LVOT pressure gradient of at least 35, 40 mm Hg, 45 mm Hg or 50 mm Hg. In some embodiments, the pressure gradient across the LVOT in the individual is measured at rest. In some embodiments, the pressure gradient across the LVOT in the individual is measured during or immediately after a Valsalva maneuver is performed. In some embodiments, the pressure gradient across the LVOT in the individual is measured post-exercise. oHCM may lead to severe symptoms of heart failure, arrhythmias, and/or death.

In certain embodiments, HCM is non-obstructive HCM.

In some embodiments, the patient additionally has one or more conditions selected from sleep apnea, hypertension, hyperlipidemia, hyperthyroidism, obesity, diabetes mellitus, glucose intolerance, alcohol use, tobacco use, prior myocardial infarction, chronic obstructive pulmonary disease, heart failure, coronary heart disease, rheumatic heart disease, valvular heart disease, nonvalvular heart disease, left ventricular hypertrophy, left ventricular diastolic dysfunction, and renal disease.

The therapies of the present disclosure may be used to treat a patient with AF with or without diastolic dysfunction (e.g., HCM). In some instances, the therapies may be used to maintain sinus rhythm (e.g., normal sinus rhythm) in a patient with AF, and/or may be used to reduce atrial fibrillation recurrence in a patient with AF.

A therapy described herein may include the step of selecting a patient with a type of atrial dysfunction as described herein (e.g., AF). In some embodiments, the patient is further selected as having a type of diastolic dysfunction as described herein (e.g., HCM).

In some embodiments, a patient treated by a therapy described herein has previously been or is being treated for the atrial dysfunction and/or diastolic dysfunction, with, for example, the standard of care for said condition(s), and has not shown adequate improvement with said treatment.

### Treatment Regimens

The Compound I therapies described herein may treat atrial dysfunction (e.g., AF) in a patient. In certain embodiments, the patient may also have diastolic dysfunction such as HCM. The patient may receive a therapy of the present disclosure for at least one month, at least six months, at least twelve months, at least one year, or longer, or until such time the patient no longer needs the treatment.

In some embodiments of the present therapies, Compound I (e.g., mavacamten or Compound A) is administered in a total oral daily dosage of about 1 mg to 50 mg. In some embodiments, the total daily dosage is about 1 mg to 50 mg. In some embodiments, the total daily dosage of Compound I is about 2 mg to 30 mg, 10 mg to 20 mg, 2 mg to 10 mg, or 2 mg to 5 mg. In some embodiments, the total daily dosage of Compound I is 2.5 mg to 15 mg. In some embodiments, the total daily dosage of Compound I is 5 mg to 15 mg. In some embodiments, the total daily dosage of Compound I is 2 mg to 15 mg. In some embodiments, the total daily dosage of Compound I is 1 mg to 15 mg. In some embodiments, the effective amount to achieve and maintain the desired blood plasma concentration of Compound I is from 2.5 to 20 mg.

In some embodiments of the present therapies, Compound I (e.g., mavacamten or Compound A) is administered in a total oral daily dosage (e.g., once daily) of 1, 2, 2.5, 5, 7.5, 10, 15, 20, or 25 mg.

The dosage used for a particular patient may be adjusted based on the patient's condition and/or the patient's unique PK profile. In some embodiments, Compound I (e.g., mavacamten or Compound A) is dosed such that its plasma concentration in the subject is between 225-600 ng/mL, 350-700 ng/mL, or 150-600 ng/mL. In certain embodiments, Compound I is dosed such that its plasma concentration in the subject is between 200-400 ng/mL. The Compound I plasma concentration may be determined by any method known in the art, such as, for example, high performance liquid chromatography (HPLC), liquid chromatography-mass spectroscopy (LC-MS such as high performance LC-MS), gas chromatography (GC), or any combination thereof.

In some embodiments, Compound I is administered to the patient at a dose whereby the LVOT within the subject decreases without a significant change in LVEF.

As used herein, administration of Compound I or a pharmaceutical composition containing Compound I (“Compound I medication”) includes self-administration by the patient himself or herself (e.g., oral intake by the patient).

In particular embodiments, Compound I is mavacamten.

### Combination Therapy

The present disclosure provides both Compound I monotherapy and combination therapy. In combination therapy, a Compound I regimen of the present disclosure is used in combination with an additional therapy regimen, e.g., a guideline-directed medical therapy (GDMT), also referred to as a standard of care (SOC) therapy, for one or more cardiac conditions exhibited by the patient, or other therapy useful for treating the relevant disease or disorder. The additional therapeutic agent may be administered by a route and in an amount commonly used for said agent or at a reduced amount, and may be administered simultaneously, sequentially, or concurrently with Compound I.

In some embodiments, Compound I is administered on top of the SOC for a condition of atrial dysfunction, such as atrial fibrillation; a condition of diastolic dysfunction, such as HCM; or both.

In certain embodiments, the patient exhibiting atrial dysfunction (e.g., atrial fibrillation) is given, in addition to the Compound I medication, another therapeutic agent for treating the atrial dysfunction. In some embodiments, the therapeutic agent is an antithrombotic agent (e.g., an anticoagulant such as a novel oral anticoagulant (NOAC)), a rate control agent, an antiarrhythmic agent (e.g., a Class Ia, Ic, or III antiarrhythmic agent), a pharmacological cardioversion agent, a RAAS inhibitor, etc. In some embodiments, the Compound I medication is administered to a patient who has had or plans to have a non-pharmacological intervention such as electrical cardioversion, left atrial appendage occlusion/excision, atrioventricular nodal ablation (e.g., with permanent ventricular pacing), AF catheter ablation, AF surgical ablation (e.g., Maze procedure), pulmonary vein ablation, or a permanent pacemaker. Any combination of the above agents and interventions is also contemplated.

In some embodiments, the Compound I medication is administered to the patient in place of an antiarrhythmic agent. The patient may have had prior treatment with an antiarrhythmic agent that is then replaced by the Compound I medication, or the patient may be treated with the Compound I medication without prior treatment with an antiarrhythmic agent.

In some embodiments, a patient with atrial dysfunction (e.g., AF) is treated with a beta blocker in addition to the Compound I medication.

In some embodiments, a patient with atrial dysfunction (e.g., AF) is treated with an anticoagulant (e.g., a NOAC) in combination with a rate control agent (e.g., a beta blocker, digoxin, and/or amiodarone) in addition to the Compound I medication.

In some embodiments, a patient with atrial dysfunction (e.g., AF) is treated with an anticoagulant (e.g., a NOAC) in combination with a rate control agent (e.g., a beta blocker, digoxin, and/or amiodarone) in addition to the Compound I medication.

In some embodiments, a patient with atrial dysfunction (e.g., AF) is treated with cardioversion (e.g., electrical cardioversion) in addition to the Compound I medication.

In some embodiments, a patient with atrial dysfunction (e.g., AF) is treated with cardioversion (e.g., electrical cardioversion) in combination with an antiarrhythmic drug (e.g., amiodarone, sotalol, or dofetilide) in addition to the Compound I medication.

In certain embodiments, the patient exhibiting diastolic dysfunction (e.g., HCM) in addition to the atrial dysfunction (e.g., AF) is given, in addition to the Compound I medication and optionally a therapeutic agent for treating the atrial dysfunction as described herein, another therapeutic agent for treating the diastolic dysfunction. In some embodiments, the therapeutic agent is a beta blocker, a calcium channel blocker (e.g., a non-dihydropyridine calcium channel blocker), disopyramide, or any combination thereof. In certain embodiments, the beta blockers and/or calcium channel blockers are selected from those described herein, in any combination.

In some embodiments, a patient with atrial dysfunction (e.g., AF) and diastolic dysfunction (e.g., HCM) is treated with surgical myectomy in addition to Compound I medication.

In some embodiments, a patient with atrial dysfunction (e.g., AF) and diastolic dysfunction (e.g., HCM) is treated with alcohol septal ablation in addition to Compound I medication.

In some embodiments, a patient with atrial dysfunction (e.g., AF) and diastolic dysfunction (e.g., HCM) is treated with an anticoagulant (e.g., an oral anticoagulant (OAC)) in combination with an antiarrhythmic agent and/or AF catheter ablation in addition to the Compound I medication.

In some embodiments, a patient with atrial dysfunction (e.g., AF) and diastolic dysfunction (e.g., HCM) is treated with amiodarone and/or disopyramide combined with a beta blocker and/or a nondihydropyridine calcium channel antagonist in addition to the Compound I medication.

In some embodiments, a patient with atrial dysfunction (e.g., AF) and diastolic dysfunction (e.g., HCM) is treated with sotalol, dofetilide, and/or dronedarone in addition to the Compound I medication.

In some embodiments, a patient with atrial dysfunction (e.g., AF) and diastolic dysfunction (e.g., HCM) is treated with an angiotensin receptor neprilysin inhibitor (ARNI) in addition to the Compound I medication. In certain embodiments, the patient is treated with sacubitril, valsartan, or Entresto® in addition to the Compound I medication.

In some embodiments, Compound I is administered to a patient with atrial dysfunction (e.g., AF) on top of the SOC for obstructive or non-obstructive HCM in combination with AF.

Suitable antiarrhythmic medications (rhythm control agents) may include, e.g., amiodarone, dronedarone, propafenone, flecainide, dofetilide, ibutilide, quinidine, procainamide, disopyramide, and sotalol. In some embodiments, the antiarrhythmic medications are of Class Ia, Ic, or III.

Suitable anticoagulants may include, e.g., warfarin, apixaban, rivaroxaban, edoxaban, and dabigatran. In some embodiments, the anticoagulants are oral anticoagulants (OACs); in certain embodiments, OACs may be administered with vitamin K antagonists. In some embodiments, the anticoagulants are non-vitamin K oral anticoagulants (NOACs).

Suitable pharmacological cardioversion agents include, e.g., flecainide, dofetilide, propafenone, ibutilide, vernakalant, etc.

Suitable rate control agents include, e.g., beta blockers, non-dihydropyridine calcium channel blockers (e.g., verapamil, diltiazem), digitalis, and amiodarone. Suitable beta blockers include, e.g., bisoprolol, carvedilol, carvedilol CR, atenolol, esmolol, propranolol, nadolol, metaprolol tartrate, and metoprolol succinate extended release (metoprolol CR/XL)).

In some embodiments, Compound I is administered in combination with lifestyle changes such as reducing alcohol or caffeine intake, quitting smoking, limiting stimulants, achieving or maintaining a healthy weight, physical activity, treating sleep apnea, and/or controlling high blood pressure and/or blood sugar levels, or any combination thereof.

If any adverse effect occurs, the patient may be treated for the adverse effect. For example, a patient experiencing headache due to the Compound I treatment may be treated with an analgesic such as ibuprofen and acetaminophen.

Any combination of the above agents and treatments is also contemplated for treating a patient in combination with the Compound I medication.

### Treatment Outcomes

The therapies of the present disclosure treat and/or ameliorate atrial dysfunction. In some embodiments, the therapies also treat and/or ameliorate diastolic dysfunction. As used herein, the terms “treat,” “treating” and “treatment” refer to any indicia of success in the treatment or amelioration of a pathology, injury, condition, or symptom related to the dysfunction, including any objective or subjective parameter such as abatement; remission; diminishing of symptoms; making the pathology, injury, condition, or symptom more tolerable to the patient; decreasing the frequency or duration of the pathology, injury, condition, or symptom; or, in some situations, delaying or preventing the onset of the pathology, injury, condition, or symptom. Treatment or amelioration can be based on any objective or subjective parameter, including, e.g., the result of a physical examination.

For example, treatment of atrial dysfunction (e.g., AF) encompasses, but is not limited to, any one or combination of: improving atrial myocyte contractility, improving atrial contractility, improving atrial cardiomyopathy, improving atrial arrhythmia (e.g., tachyarrhythmia), reducing AF recurrence, reducing AF burden, preventing incident AF, maintaining sinus rhythm (e.g., after cardioversion), restoring sinus rhythm (e.g., in combination with cardioversion), decreasing left atrial volume, increasing left atrial emptying fraction, increasing left atrial function index, preventing conversion of α-MHC to β-MHC in the LA, and alleviating or preventing the symptoms of atrial dysfunction. Symptoms of atrial dysfunction (e.g., AF) may include, e.g., heart palpitations, tachycardia, fatigue, dizziness, weakness, chest discomfort, reduced exercise capacity, increased urination, shortness of breath, angina, presyncope, syncope, sleeping difficulties, confusion, and psychosocial distress.

Treatment of diastolic dysfunction encompasses, but is not limited to, any one or combination of improving the cardiac functions of the patient and alleviating or preventing the symptoms of diastolic dysfunction (especially during exercise, including walking or stair climbing). Symptoms of diastolic dysfunction such as HCM may include, e.g., shortness of breath (especially during exercise), chest pain (especially during exercise), fainting (especially during or just after exercise), sensation of rapid, fluttering or pounding heartbeats, atrial and ventricular arrhythmias, heart murmur, hypertrophied and non-dilated left ventricle, thickened heart muscle, thickened left ventricular wall, elevated pressure gradient across left ventricular outflow tract (LVOT), elevated post-exercise LVOT gradient, and high left ventricular ejection fraction (LVEF).

In some embodiments, the therapies of the present disclosure reduce AF burden and/or AF recurrence in a patient (e.g., a patient from a population described herein). AF burden and/or AF recurrence may be reduced by 10% or greater. In some embodiments, AF burden and/or AF recurrence are reduced by 5%, 10%, 15%, 20%, 25%, 30%, 35%, 40%, 45%, 50%, 55%, 60%, 65%, 70%, 75%, 80%, 85%, 90%, or 95% or greater, or 100%. In some embodiments, the percentage of time the patient spends in AF during a monitoring period is reduced by 5%, 10%, 15%, 20%, 25%, 30%, 35%, 40%, 45%, 50%, 55%, 60%, 65%, 70%, 75%, 80%, 85%, 90%, or 95% or greater, or 100%. In some embodiments, the therapies reduce the duration of a patient's longest AF episode, or the number of AF episodes during a monitoring period, e.g., by 5%, 10%, 1₅%, 20%, 25%, 30%, 35%, 40%, 45%, 50%, 55%, 60%, 65%, 70%, 75%, 80%, 85%, 90%, or 95% or greater, or 100%. In some embodiments, the monitoring period may be 24 hours, 1 week, 2 weeks, 1 month, 2 months, 3 months, 4 months, 5 months, 6 months, 1 year, or more.

In some embodiments, the therapies of the present disclosure maintain sinus rhythm (e.g., normal sinus rhythm) in a patient (e.g., a patient from a population described herein). In certain embodiments, the patient has been treated with or will be treated with cardioversion (e.g., electrical cardioversion). In some embodiments, the therapies of the present disclosure, in combination with cardioversion (e.g., electrical cardioversion), restore sinus rhythm (e.g., normal sinus rhythm) in a patient. In some embodiments, sinus rhythm is maintained for at least one, two, three, four, five, six, or seven days; at least one, two, three, or four weeks; at least one, two, three, four, five, six, nine, or twelve months; at least 1, 2, 3, 4, 5, 6, 7, 8, 9, 10 years; or longer; or until such time that the patient no longer needs the treatment.

In some embodiments, the therapies of the present disclosure reduce the risk of, or delay the incidence of, myocardial infarction, ventricular arrhythmia, heart failure, chronic kidney disease, end-stage renal disease, sudden cardiac death, or all-cause death in a patient.

In some embodiments, the therapies of the present disclosure improve the patient's quality of life, as measured by the 6-Month Walk Test (6-MWT), Kansas City Cardiomyopathy Questionnaire (KCCQ), Atrial Fibrillation Effect on Quality-of-Life (AFEQT) measure, and/or Mayo AF-Specific Symptom Inventory (MAFSI).

In some embodiments, the therapies of the present disclosure prevent or delay tachycardia-induced cardiomyopathy in patients exhibiting atrial fibrillation.

In some embodiments, the therapies of the present disclosure prevent or delay incident AF (initial occurrence of AF) in patients. Additionally or alternatively, the therapies may prevent or delay AF recurrence in patients.

Pharmacodynamic (PD) parameters that can be used to measure the atrial functions of a patient are shown in Table 3 below. These PD parameters are routinely used by clinicians and can be measured by standard transthoracic echocardiogram.

TABLE 3
Transthoracic Echocardiography (TTE) Parameters

| Abbreviation | Parameter |
|---|---|
| LAEF | Left Atrial Emptying Fraction |
| LAₘₐₓVi | Left Atrial Maximum Volume Index |
| LAₘᵢₙVi | Left Atrial Minimum Volume Index |
| LAFI | Left Atrial Functional Index |

In some embodiments, the therapies of the present disclosure:
increase LAEF in the patient by 5%, 10%, 15%, 20%, 25%, 30%, 35%, 40%, 45%, 50%, or more;
decrease LAₘᵢₙVi in the patient by 5%, 10%, 15%, 20%, 25%, 30%, 35%, 40%, 45%, 50%, or more;
decrease LAₘₐₓVi in the patient by 5%, 10%, 15%, 20%, 25%, 30%, 35%, 40%, 45%, 50%, or more; and/or
increase LAFI in the patient by 5%, 10%, 15%, 20%, 25%, 30%, 35%, 40%, 45%, 50%, or more.

In some embodiments, the therapies of the present disclosure reduce biomechanical remodeling of the left atrium, such as increased maximal force and slowed cross-bridge formation rates (e.g., in a patient with atrial dysfunction such as AF and/or diastolic dysfunction such as HCM).

In some embodiments, the therapies of the present disclosure prevent, slow, and/or reverse the conversion of α-myosin heavy chain (MHC) to β-MHC in the left atrium of a patient by 20%, 25%, 30%, 35%, 40%, 50%, 60%, 70%, 80%, 90%, or more (e.g., in a patient with atrial dysfunction such as AF and/or diastolic dysfunction such as HCM).

In some embodiments, the therapies of the present disclosure
reduce cardiac troponin T (cTnT leakage) by 5%, 10%, 15%, 20%, 25%, 30%, 35%, 40%, 50%, 60%, 70%, 80%, 90%, or more; and/or
reduce incidence of cTNT by 5%, 10%, 15%, 20%, 25%, 30%, 35%, 40%, 45%, 50%, 60%, 70%, 80%, 90%, or more (e.g., in a patient with atrial dysfunction such as AF and/or diastolic dysfunction such as HCM).

In certain embodiments, the cTnT is high sensitivity cTnT.

In some embodiments, the therapies of the present disclosure downregulate BNP and/or pro-fibrotic genes (e.g., SPP1, NPPB, TGF-β1, TGF-β2, FN1, CCN2, or any combination thereof) by 5%, 10%, 15%, 20%, 25%, 30%, 35%, 40%, 50%, 60%, 70%, 80%, 90%, or more.

In some embodiments, the therapies of the present disclosure reduce mortality in a patient by 5%, 10%, 15%, 20%, 25%, 30%, 35%, 40%, 50%, 60%, 70%, 80%, 90%, or more (e.g., in a patient with atrial dysfunction such as AF and/or diastolic dysfunction such as HCM).

The patient treated with a therapy described herein may exhibit New York Heart Association (NYHA) Class I, II, III, or IV heart failure, as defined in Table 2 below. In certain embodiments, the patient has NYHA Class II-IV heart failure. In some embodiments, the therapies of the present disclosure improve, stabilize, or delay worsening of the NYHA functional classification of the patient.

TABLE 2
New York Heart Association (NYHA) Classes of Heart Failure

| Class | Patient Symptoms |
|---|---|
| I | No limitation of physical activity. Ordinary physical activity does |
| | not cause undue fatigue, palpitation, dyspnea (shortness of breath). |
| II | Slight limitation of physical activity. Comfortable at rest. Ordinary |
| | physical activity results in fatigue, palpitation, dyspnea (shortness |
| | of breath). |
| III | Marked limitation of physical activity. Comfortable at rest. Less |
| | than ordinary activity causes fatigue, palpitation, or dyspnea. |
| IV | Unable to carry on any physical activity without discomfort. |
| | Symptoms of heart failure at rest. If any physical activity is |
| | undertaken, discomfort increases. |

The present therapies may reduce the risk of cardiovascular death, and/or the risk, frequency, or duration of hospitalization/urgent care visits, for a patient population described herein. The hospitalization and urgent care visits may be for atrial dysfunction as described herein, diastolic dysfunction as described herein, or both. In some embodiments, “reducing the risk” of an event means increasing the time to the event by at least 10% (e.g., at least 15%, 20%, 30%, 40%, 50%, 60%, 70%, 80%, 90%, 100%, or more). The risk can be relative risk or absolute risk. In some embodiments, the present therapies reduce the frequency of hospitalization and urgent care visits by at least 10% (e.g., at least 15%, 20%, 30%, 40%, 50%, 60%, 70%, 80%, 90%, or 100%). In some embodiments, the present therapies reduce the duration of hospitalization by at least 10% (e.g., at least 15%, 20%, 30%, 40%, 50%, 60%, 70%, 80%, 90%, or 100%).

### Articles of Manufacture and Kits

The present invention also provides articles of manufacture, e.g., kits, comprising one or more dosages of the Compound I medication, and instructions for patients (e.g., for treatment in accordance with a method described herein). The articles of manufacture may also contain an additional therapeutic agent in the case of combination therapy. Compound I tablets or capsules may be blistered and then carded, produced with, for example, 5-20 tablets per blister card; each tablet or capsule may contain 1, 2, 2.5, 5, 7.5, 10, 15, 20, or 25 mg of Compound I, and such blister card may or may not additionally include a loading dose tablet or capsule. The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Chronic Treatment with Compound A in a Mini-Pig Model of Inherited Hypertrophic Cardiomyopathy

This Example describes an in vivo study that evaluates the chronic effects of Compound A, in a genetic large-animal model of HCM.

### Materials and Methods

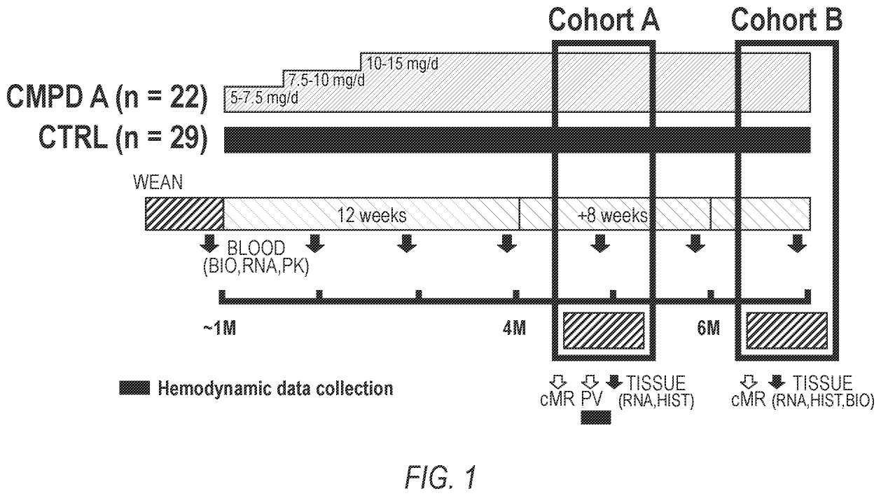

Young (~1 month) cloned Yucatan mini-pigs with a heterozygous MYH7 R403Q mutation (“HCM pigs”) were treated and evaluated as shown in FIG. 1. The pigs were randomly assigned to one of two arms: untreated controls (CTRL, n=29) or daily Compound A (n=22; PO); untreated wild-type pigs (WT) served as disease controls. The pigs treated with Compound A were divided into two cohorts: A and B. Treated animals received progressively increasing Compound A doses (5, 7.5, 10, and 15 mg/day PO) to account for weight gain. After 14 weeks of treatment, pigs underwent in vivo cardiac MRI (cMR) imaging, including T1 mapping and extracellular volume (ECV) assessments. In a subset of pigs (Cohort B), biomechanical studies were performed in skinned left-ventricular (LV) and left-atrial (LV) fibers.

### Results

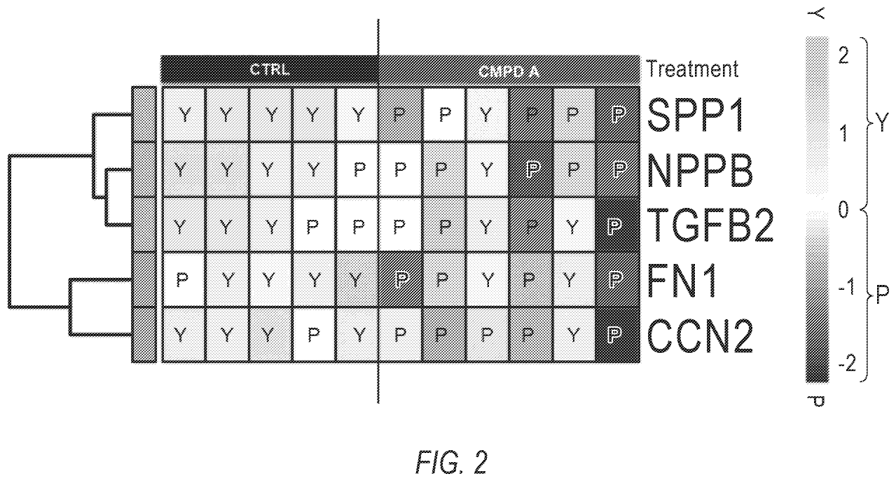

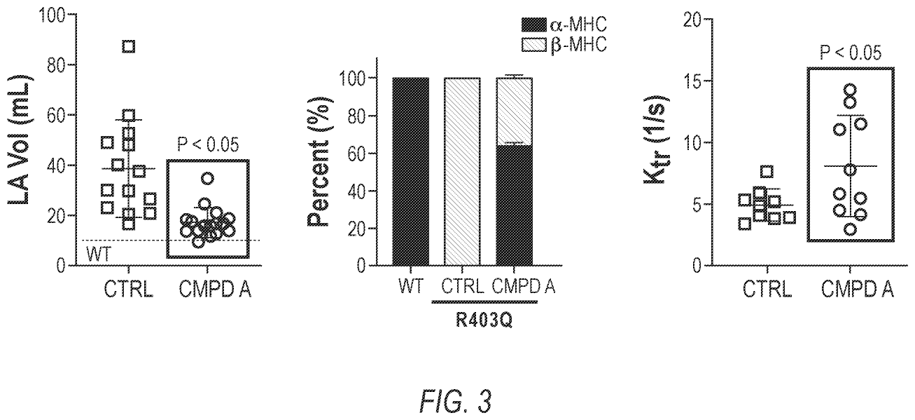

In HCM pigs, Compound A treatment decreased mortality (9.0* vs. 37.9% in CTRL, p<0.05). Compound A blunted cTnT leakage, reducing both absolute values (21.2±3.2* vs. 34.0±4.3 ng/L in CTRL) and the incidence of cTnT>20 ng/L (36* vs. 81% in CTRL). Moreover, Compound A caused downregulation of BNP and pro-fibrotic genes (e.g., NPPB, TGF-β1 and β2, SPP1, FN1, and CCN2) that were elevated in the CTRL group (FIG. 2). Treated pigs had smaller LA volumes (16±1* vs. 29±4 mL in CTRL) with lower LV T1-times and ECV (27±1* vs. 32±2% in CTRL). LA fibers from untreated HCM pigs showed biomechanical remodeling characteristic of chronic overload: increased maximal force (21.8±1.5 #vs. 14.7±1.4 mN/mm2 in WT) and slowed cross-bridge formation rates (Kₜᵣ: 4.9±0.4 #vs. 6.6±0.6 s−1 in WT) consistent with a switch towards slow-myosin isoforms. Compound A prevented this remodeling (e.g., Kₜr: 8.1±1.3 s−1*), preserving normal LA (fast) myosin content and isometric tension redevelopment dynamics (FIG. 3). *, #: P<0.05 vs. CTRL or WT.

As demonstrated by the above data, chronic direct myosin attenuation with Compound A prevented left atrial remodeling, a known prognostic indicator in HCM. Chronic treatment also reduced cardiac troponin leakage characteristic of HCM, and decreased mortality. Taken together, these observations show potential salutary effects beyond obstruction relief in HCM.

### Example 2: Biochemical Properties of Compound A and Mavacamten

This Example describes a study that demonstrates that Compound A mirrors the functional properties of mavacamten.

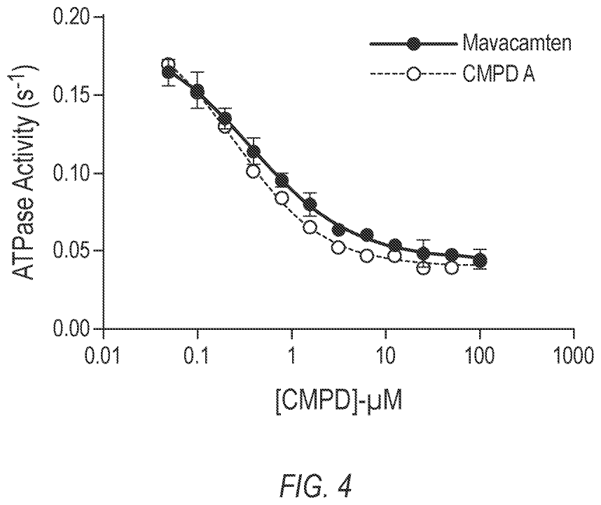

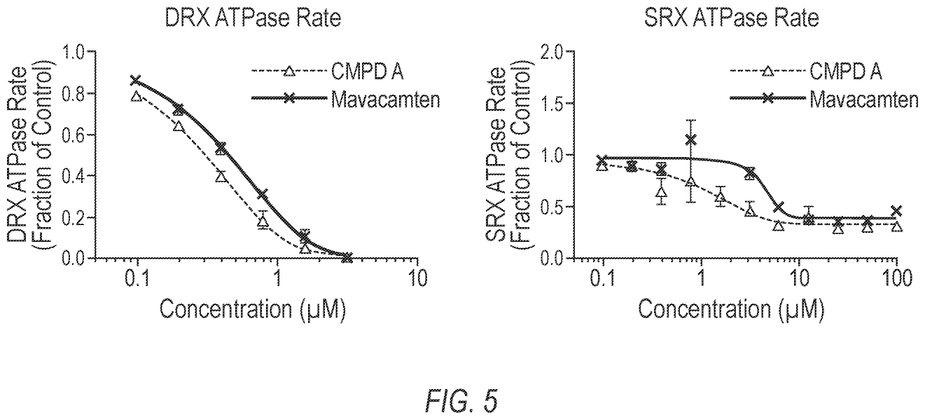

Mavacamten is a cardiac-specific, small-molecule allosteric modulator of B-cardiac myosin that reversibly inhibits its binding to actin. Compound A is a mavacamten surrogate in which a hydrogen has been substituted with fluorine. To compare the activity of Compound A and mavacamten, both compounds were tested in bovine cardiac myofibrils for inhibition of myosin ATPase. At pCa 6.0, Compound A and mavacamten exhibited similar inhibition of myosin ATPase activity over the tested concentration range (FIG. 4). Comparative studies of Compound A and mavacamten in bovine cardiac synthetic myosin filaments showed similar disordered relaxed state (DRX) and super-relaxed state (SRX) ATPase rates (as a fraction of control) for the two compounds over a range of concentrations (FIG. 5). Due to the above-described similarities, mavacamten is expected to perform similarly to Compound A in the treatments described herein.

What is claimed is:

1. A method of treating atrial dysfunction in a patient in need thereof, comprising administering to the patient a therapeutically effective amount of a compound of formula (I), 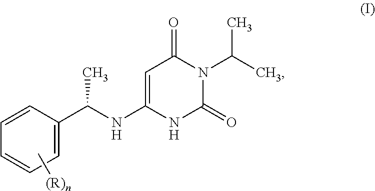
or a pharmaceutically acceptable salt thereof, wherein n is 0, 1, 2, 3, 4, or 5, and R is a halogen, optionally a fluorine, further optionally wherein the patient exhibits atrial fibrillation.

2. A method of treating atrial cardiomyopathy in a patient in need thereof, comprising administering to the patient a therapeutically effective amount of a compound of formula (I), 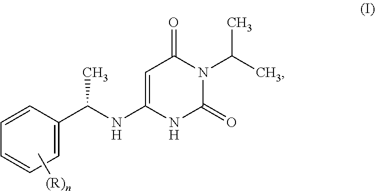
or a pharmaceutically acceptable salt thereof, wherein n is 0, 1, 2, 3, 4, or 5, and R is a halogen, optionally a fluorine, further optionally wherein the patient exhibits atrial fibrillation.

3. A method of treating atrial tachyarrhythmia in a patient in need thereof, comprising administering to the patient a therapeutically effective amount of a compound of formula (I), 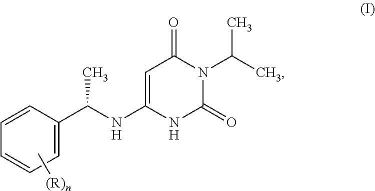
or a pharmaceutically acceptable salt thereof, wherein n is 0, 1, 2, 3, 4, or 5, and R is a halogen, optionally a fluorine, further optionally wherein the patient exhibits atrial fibrillation.

4. A method of treating atrial fibrillation in a patient in need thereof, comprising administering to the patient a therapeutically effective amount of a compound of formula (I) 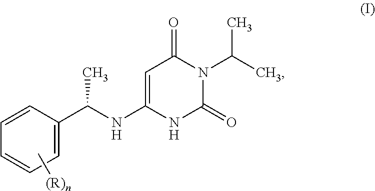
or a pharmaceutically acceptable salt thereof, wherein n is 0, 1, 2, 3, 4, or 5, and R is a halogen, optionally a fluorine.

5. A method of reducing atrial fibrillation recurrence in a patient in need thereof, comprising administering to the patient a therapeutically effective amount of a compound of formula (I), 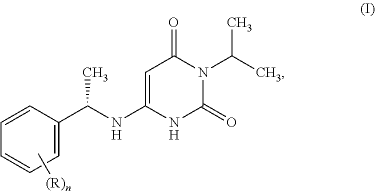
or a pharmaceutically acceptable salt thereof, wherein n is 0, 1, 2, 3, 4, or 5, and R is a halogen, optionally a fluorine, further optionally wherein atrial fibrillation recurrence is reduced by 10% or greater.

6. A method of reducing atrial fibrillation burden in a patient in need thereof, comprising administering to the patient a therapeutically effective amount of a compound of formula (I) 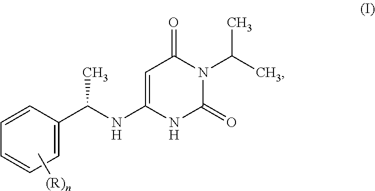
or a pharmaceutically acceptable salt thereof, wherein n is 0, 1, 2, 3, 4, or 5, and R is a halogen, optionally a fluorine, further optionally wherein atrial fibrillation burden is reduced by 10% or greater.

7. A method of reducing the duration of an atrial fibrillation episode in a patient in need thereof, comprising administering to the patient a therapeutically effective amount of a compound of formula (I) 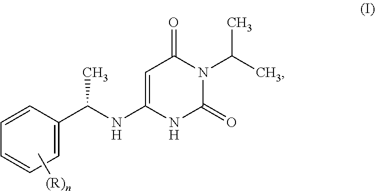
or a pharmaceutically acceptable salt thereof, wherein n is 0, 1, 2, 3, 4, or 5, and R is a halogen, optionally a fluorine, further optionally wherein the duration of the episode is reduced by 10% or greater.

8. A method of reducing the number of atrial fibrillation episodes during a monitoring period in a patient in need thereof, comprising administering to the patient a therapeutically effective amount of a compound of formula (I) 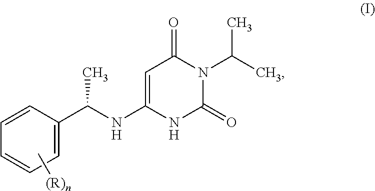
or a pharmaceutically acceptable salt thereof, wherein n is 0, 1, 2, 3, 4, or 5, and R is a halogen, optionally a fluorine, further optionally wherein the number of atrial fibrillation episodes is reduced by 10% or greater.

9. A method of maintaining sinus rhythm in a patient in need thereof, comprising administering to the patient a therapeutically effective amount of a compound of formula (I) 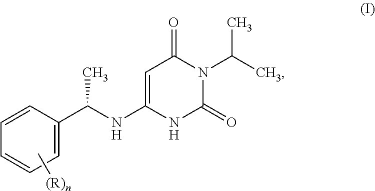
or a pharmaceutically acceptable salt thereof, wherein n is 0, 1, 2, 3, 4, or 5, and R is a halogen, optionally a fluorine, further optionally wherein the patient has sustained atrial tachyarrhythmia for 12 months or less prior to the administering step, further optionally wherein the atrial tachyarrhythmia is atrial fibrillation.

10. A method of restoring sinus rhythm in a patient exhibiting atrial tachyarrhythmia, comprising administering to the patient a therapeutically effective amount of a compound of formula (I) 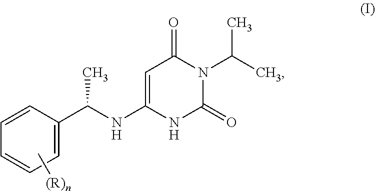
or a pharmaceutically acceptable salt thereof, wherein n is 0, 1, 2, 3, 4, or 5, and R is a halogen, optionally a fluorine, further optionally wherein the cardioversion is electrical cardioversion and further optionally wherein the atrial tachyarrhythmia is atrial fibrillation.

11. A method of preventing or slowing the conversion of α-myosin heavy chain (MHC) to β-MHC in the left atrium of a patient, comprising administering to the patient a therapeutically effective amount of a compound of formula (I) 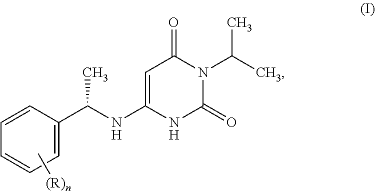
or a pharmaceutically acceptable salt thereof, wherein n is 0, 1, 2, 3, 4, or 5, and R is a halogen, optionally a fluorine, further optionally wherein the patient exhibits atrial fibrillation.

12. A method of reversing the conversion of α-myosin heavy chain (MHC) to β-MHC in the left atrium of a patient, comprising administering to the patient a therapeutically effective amount of a compound of formula (I) 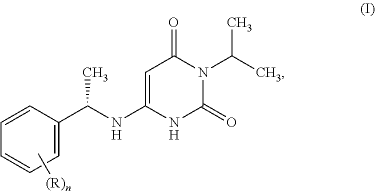
or a pharmaceutically acceptable salt thereof, wherein n is 0, 1, 2, 3, 4, or 5, and R is a halogen, optionally a fluorine, further optionally wherein the patient exhibits atrial fibrillation.

13. The method of any one of claims 1-12, wherein n is 0.

14. The method of any one of claims 1-12, wherein R is fluorine.

15. The method of claim 14, wherein n is 1.

16. The method of claim 15, wherein R is a fluorine at position 3.

17. The method of any one of claims 1-16, wherein the patient also exhibits diastolic dysfunction.

18. The method of claim 17, wherein the diastolic dysfunction is hypertrophic cardiomyopathy (HCM).

19. The method of claim 18, wherein the hypertrophic cardiomyopathy is obstructive hypertrophic cardiomyopathy (oHCM).

20. The method of claim 19, wherein the patient has at least a 30 mm Hg pressure gradient across the left ventricular outflow tract (LVOT) at rest, during or immediately after a Valsalva maneuver, or post-exercise.

21. The method of claim 18, wherein the hypertrophic cardiomyopathy is non-obstructive hypertrophic cardiomyopathy (nHCM).

22. The method of any one of claims 1-21, wherein the patient has heart failure and a diagnosis of any one of NYHA Class II-IV.

23. The method of any one of claims 1-22, wherein the patient exhibits atrial fibrillation; and the therapeutically effective amount of Compound I alleviates one or more symptoms of HCM, maintains sinus rhythm, reduces atrial fibrillation recurrence, and/or prevents incident atrial fibrillation in the patient.

24. A method of preventing tachycardia-induced cardiomyopathy in a patient exhibiting atrial fibrillation, comprising administering to the patient a therapeutically effective amount of a compound of formula (I) 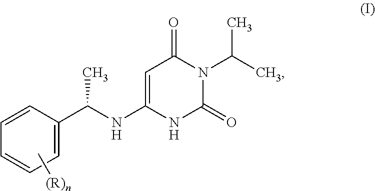
or a pharmaceutically acceptable salt thereof, wherein n is 0, 1, 2, 3, 4, or 5, and R is a halogen, optionally a fluorine.

25. The method of any one of claims 1-24 wherein the patient has sustained the tachyarrhythmia or atrial fibrillation for a duration of 12 months or less prior to the administering step.

26. The method of any one of claims 1-25, wherein the patient has an atrial fibrillation burden of 2-70%.

27. The method of any one of claims 1-26, wherein the Compound I is administered to the patient orally.

28. The method of any one of claims 1-27, wherein the patient has undergone an electrical cardioversion before or after the administering step, optionally where the electrical cardioversion is performed no more than 24 hours before or after the administering step.

29. The method of any one of claims 1-28, further comprising administering to the patient an additional medication for improving cardiovascular conditions in the patient.

30. The method of any one of claims 1-29, wherein the method results in any one or combination of the following:
a) reduced risk of urgent outpatient intervention for atrial dysfunction, diastolic dysfunction, or both;
b) improved quality of life as measured through 6-MWT or KCCQ;
c) improved exercise capacity;
d) improvement in a patient's NYHA classification;
e) delay in clinical worsening;
f) reduction in severity of cardiovascular-related symptoms;
g) increased left atrial ejection fraction (LAEF);
h) decreased left atrial volume (LAₘᵢₙVI);
i) improved left atrial function index (LAFI);
j) reduced cTnT leakage;
k) decreased BNP; and
l) decreased expression of pro-fibrotic genes.

31. The method of any one of claims 1-30, wherein the method results in reduced cardiovascular death or hospitalization.

32. The method of any one of claims 1-30, wherein the method results in reduced stroke, embolism, or heart failure.

33. A compound of formula (I) 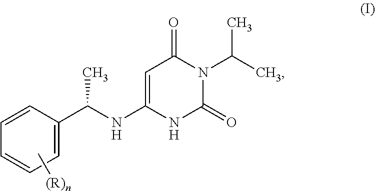
or a pharmaceutically acceptable salt thereof, wherein n is 0, 1, 2, 3, 4, or 5, and R is a halogen, optionally a fluorine, or a pharmaceutical composition comprising the compound and a pharmaceutically acceptable excipient, for use in treating a disease according to any one of claims 1-32.

34. Use of a compound of formula (I) 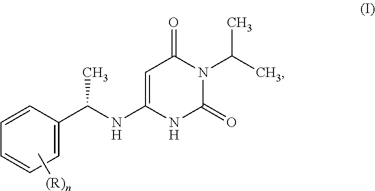
or a pharmaceutically acceptable salt thereof, wherein n is 0, 1, 2, 3, 4, or 5, and R is a halogen, optionally a fluorine, in the manufacture of a medicament for the treatment of a disease according to any one of claims 1-32.
